(19) United States
(12) Patent Application Publication
(10) Pub. No.: US 2025/0312417 A1
(43) Pub. Date: Oct. 9, 2025

(54) Methods for Mitigating Lung Injury in Conjunction with Exposure to Radiation and/or Radiation or Radiomimetic Treatments

(71) Applicant: Janssen Pharmaceutica NV, Beerse (BE)

(72) Inventors: Gary M. EICHENBAUM, Belle Mead, NJ (US); Chandan GUHA, Scarsdale, NY (US); Jeb ENGLISH, Bronx, NY (US); Weng-Lang YANG, Bronx, NY (US)

(21) Appl. No.: 18/867,368

(22) PCT Filed: May 19, 2023
(86) PCT No.: PCT/US2023/067214

§ 371 Date: Nov. 19, 2024

Related U.S. Application Data
(60) Provisional application No. 63/344,285, filed on May 20, 2022.

(51) Int. Cl. A61K 38/19; A61N 5/10

(52) U.S. Cl. CPC A61K 38/196; A61N 5/10

(57) ABSTRACT

Methods and kits for mitigating lung injury in a subject treated with a targeted radiation therapy or otherwise exposed to radiation are described. In particular, an effective amount of a thrombopoietin mimetic, such as RWJ-800088 or romiplostim, is used to mitigate the radiation-induced lung injury.

### CROSS-REFERENCE TO RELATED APPLICATIONS

This application claims priority to U.S. Provisional Application No. 63/344,285, filed on May 20, 2022, the disclosure of which is incorporated herein by reference in its entirety.

### REFERENCE TO SEQUENCE LISTING SUBMITTED ELECTRONICALLY

This application contains a sequence listing, which is or will be submitted electronically via PatentCenter as an XML formatted sequence listing with a file name “004852-213WO1 Sequence Lisitng.xml”, created on May 15, 2023, and having a size of 13,348 bytes. The sequence listing submitted via PatentCenter is or will be part of the specification and is herein incorporated by reference in its entirety.

### FIELD OF THE INVENTION

This invention relates to methods and kits for mitigating lung injury in a subject in need thereof. In particular, this invention relates to methods comprising administering to the subject an effective amount of a thrombopoietin (TPO) mimetic, as well as kits containing a pharmaceutical composition comprising an effective amount of a TPO mimetic and a pharmaceutically acceptable carrier. The TPO can be administered alone or in combination with other active agents to promote beneficial effects

### BACKGROUND OF THE INVENTION

Radiotherapy is an indispensable strategy for cancer treatment. About 60-70% of patients with malignancies receive radiation therapy or radiotherapy which can cure many tumors and the cure rates of radiotherapy on early tongue cancer, nasopharynx, laryngeal cancer, esophageal cancer and cervical cancer are about 90% [Hogle W P, Semin Oncol Nurs, 22 (4): 212-220, (2006)]. However, when killing the tumor, radiotherapy can cause off-target effect on normal tissue (including non-cancerous tissue inside the radiation shield and distant tissues such as bone marrow), which limits the efficacy of radiotherapy. Although measures including the updating of equipment to improve radiotherapy accuracy, the cooperative usage of radiosensitizers and the combination of radiotherapy with chemotherapy have been tried, the results remain unsatisfactory. Normal tissues are not able to tolerate radiation-induced toxicity, which prevents use of higher doses of radiotherapy in clinical applications. Radiation-induced lung injury is the most common clinical complication post-radiotherapy. In severe cases, it may endanger the patients' lives, especially those who suffer from lung tumors, esophageal tumors, breast tumors and mediastinal tumors.

Radiation-induced lung injury (RILI) includes radiation pneumonitis in the early stage and radio-pulmonary fibrosis in the late stage. Such injury not only undermines the control of tumors, but also seriously affects the quality of life of the patients. Respiratory failure is one of the leading causes of death in RILI. In addition, local hypoxia, inflammatory response, angiogenesis, local microenvironmental changes and immunosuppression caused by RILI will promote tumor recurrence, invasion and metastasis [van den Brenk, H A et al, Br J Radiol, 47 (558): p. 332-336, (1974)]. Thus, it is particularly important to manage RILI in the clinic. The lack of effective drugs leads to empirical use of high-dose glucocorticoid and anti-inflammatory drugs. These measures often not only fail to improve the therapeutic effect of radiotherapy, but also cause many side effects, such as immunosuppression. Additionally, pulmonary immunosuppressive microenvironment spurs the risk of tumor recurrence and metastasis. There is thus a need to identify an agent that can prevent and/or treat RILI.

It is generally accepted that the tumor-killing effect of radiotherapy is due to radiation-induced DNA damage and the production of free radicals inside tumor cells [Muruve D A et al, Nature, 452 (7183): 103-107, (2008)]. Subsequently, DNA fragments and reactive oxygen species (ROS) trigger inflammation, during which activated macrophages synthesize and secrete a large amount of inflammatory cytokines, such as TNF-α, IL-1β, IL-8, etc. High levels of TNF-α and fibronectin together can cause the initial acute pneumonitis, which can also promote the proliferation of fibroblasts and stimulate fibroblasts to secrete excess collagen at the same time. Radiation-induced oxidative damage in the pulmonary capillary endothelial cells, including DNA breakage, cell death, and the increase of reactive oxygen species/reactive nitrogen species (ROS/RNS), causes the accumulation, transcription and up-regulated activity of hypoxia-inducible factor (HIF) in tumor cells [Lerman, O Z, et al., Blood, 116 (18): 3669-3676, (2010)]. Under hypoxia, vascular endothelial cells (ECs) produce a large amount of chemokine stromal cell-derived factor (SDF), which binds to chemokine receptor CXCR4 and recruits BMDCs to inflammatory lesions [Du, R., et al., Cancer Cell, 13 (3): 206-220, (2008)]. Studies have shown that bone marrow derived cells (BMDCs) are crucial for the formation and growth of tumor neovascularization. The changes of the microenvironment provide a favorable condition for tumor recurrence and metastasis.

Although existing drugs for the prevention and treatment of RILI have some protective effects, in many cases they do not work sufficiently and/or can inhibit the therapeutic efficacy of radiotherapy. There is therefore a need for a drug that can prevent RILI without affecting the efficacy of radiotherapy.

Although significant efforts are made in the clinic to limit the volume of normal lung tissue exposed to radiation when treating a lung tumor due to the radiosensitivity of the lung, to adequately treat a patient's cancer it may not be possible to avoid exposure of normal tissue to radiation and long-term lung toxicity can occur. In addition, cases of accidental radiation exposures have been described, including patients receiving unintended thoracic irradiation during treatment of breast, lung and other cancers; the subsequent lung complications have, in some instances, led to patient deaths. Such outcomes underscore the critical role played by the lung in both early and late radiation lethality. Thus, there is a growing realization that, in addition to countermeasures for the classically recognized components of acute radiation syndrome (ARS), such as neutropenia and thrombocytopenia, agents are also needed that are specifically targeted at the pulmonary response, particularly in the context of total body irradiation (TBI) such as might be anticipated as part of a radiation incident.

Radiation-induced pulmonary syndrome is a delayed lethal event from accidental or intentional exposure to irradiation in case of nuclear accidents or terrorism. In the event of a nuclear accident or deliberate attack resulting in a large population exposure to ionizing radiation, victims will need to be triaged according to the severity of acute radiation illness. Radiation-induced bone-marrow syndrome and gastrointestinal (GI) syndrome occur at lower doses of radiation and have an earlier onset than does radiation-induced pulmonary syndrome. Although acute lung injury is not an early event compared to radiation-induced gastrointestinal and hematologic disorder, successful treatment of gastrointestinal and hematologic syndromes might not rescue patients completely as mortality from respiratory distress at a later time point is always an issue.

Furthermore, many victims at risk for development of chronic injury will not be symptomatic for months to years after exposure. Therefore, it is necessary to develop a therapeutic strategy that is effective against the onset of symptomatic injury.

Two phases of radiation lung injury have been described. Acute radiation pneumonopathy (pneumonitis) can occur from several weeks to 6 months post-irradiation. If a large volume of lung has been affected, this phase can be life threatening. In late radiation-induced lung injury, occurring months to years after irradiation, the number of inflammatory cells decreases and deposition of collagen occurs, resulting in irreversible lung fibrosis.

The present invention addresses the need to prevent and/or treat RILI.

### BRIEF SUMMARY OF THE INVENTION

It is now discovered that thrombopoietin (TPO) mimetics can mitigate lung injury in a subject in need thereof. For example, it is found that TPO mimetics have significant mitigating effects on targeted radiation-induced lung injury (RILIs). It is expected that TPO mimetics can have a significant effect if used alone or together with the administration of other active agents.

Thrombopoietin (TPO) is a growth factor that is synthesized and secreted by the liver. In addition to acting as a humoral growth factor that stimulates the proliferation and differentiation of megakaryocytes through the thrombopoietin receptor (TPO-R or c-Mpl), recombinant human TPO (rhTPO) has been shown to promote platelet activation and liver endothelial cell growth and migration in vitro (Cardier et al., Blood, 1998, 91:923-929).

Accordingly, in one general aspect, the application relates to a method of mitigating RILI in a subject in need thereof, the method comprising: administering to the subject an effective amount of a thrombopoietin (TPO) mimetic, preferably the TPO mimetic comprises the amino acid sequence of SEQ ID NO:1, more preferably the TPO mimetic is RWJ-800088 or romiplostim.

In certain embodiments, the TPO mimetic is administered to the subject in combination with another active agent. The TPO mimetic can be administered to the subject before, after, or simultaneously with the other active agents.

In certain embodiments, the subject in need of a treatment of the application is a subject treated with a radiation therapy, preferably a targeted radiation therapy, which may result in RILI, such as a targeted radiation therapy for a lung disease, preparative irradiation for bone marrow transplant, or targeted radiation therapy for a esophageal cancer. The TPO mimetic can be administered to the subject before, after, or simultaneously with the radiation therapy. In certain embodiments, the TPO mimetic is administered to the subject at least about 7 days before to about 7 days after, preferably at least about 24 hours before to at least about 24 hours after the subject is administered with a dose of radiation. In certain embodiments, the TPO mimetic is administered to the subject at 24 hours before the subject is administered with a dose of radiation. In some embodiments, the TPO mimetic is administered 24 to 2 hours before or after a dose of radiation. In other embodiments, the TPO mimetic is administered 1 minute to 2 hours before or after a dose of radiation. In some embodiments, the TPOm is administered 2 to 24 hours after a radiation dose.

In certain embodiments, the subject is treated with targeted radiation, preferably to the lung, at a dose of 5-70 Gray (Gy) in 1 to 10 fractions.

In certain embodiments, the effective amount of the TPO mimetic for humans is about 1 to about 10 g/kg, more preferably about 3 μg/kg to about 5 μg/kg of body weight of the subject. In preferred embodiments, the effective amount of the TPO mimetic is about 1 μg/kg of body weight of the subject. In certain preferred embodiments, the effective amount of the TPO mimetic is about 3 μg/kg of body weight of the subject when administered subcutaneously or intravenously. An effective dose for humans can be determined after determining an effective dose for mice by dividing by 100 based on observed differences in potency between species where, for example, a 3 mg/kg dose in mice and a 0.003 mg/kg dose in humans both produce approximately 3× transient elevation of platelets.

In certain embodiments, the effective amount of the TPO mimetic is administered to the subject by intravenous, intramuscular, or subcutaneous injection. In preferred embodiments, the TPO mimetic is administered by subcutaneous injection.

In another general aspect, the application relates to a kit for mitigating RILI in a subject in need thereof. The kit comprises a pharmaceutical composition comprising an effective amount of a TPO mimetic and a pharmaceutically acceptable carrier for mitigating the RILI. Optionally, the kit further comprises, administration with at least one additional therapeutic agent. Optionally the kit further comprises, a device or a tool for administering the TPO mimetic to the subject. Preferably, the kit comprises a TPO mimetic having the amino acid sequence of SEQ ID NO:1, more preferably the TPO mimetic of RWJ-800088 or romiplostim.

In another general aspect, the application relates to a method for treating radiation pneumonitis in a subject in need thereof, comprising administering to the subject a thrombopoietin (TPO) mimetic comprising the amino acid sequence of SEQ ID NO: 1, preferably the TPO mimetic is RWJ-800088 or romiplostim, in an amount effective to treat the radiation pneumonitis.

In certain embodiments, the subject is treated with a targeted radiation therapy for a lung disease or the subject is treated with a preparative irradiation for bone marrow transplant.

In certain embodiments, the subject is treated with the targeted radiation therapy at a dose of 5-70 Gray (Gy) in 1 to 10 fractions.

In certain embodiments, the subject is treated for a lung tumor or a lung metastasis, preferably a lung cancer.

In certain embodiments, the TPO mimetic is administered to the subject 7 days before to 7 days after; 2 days before to 2 days after; 24 hours before to 24 hours after, preferably about 2 hours to 24 hours before or after, the subject is administered a dose of radiation.

In certain embodiments, the TPO mimetic is administered to the subject 2 hours to 36 hours or 1 day before the subject is administered a dose of radiation.

In certain embodiments, the TPO mimetic is administered to the subject by any one of intravenous, intramuscular, intracutaneous, or subcutaneous injection.

In certain embodiments, the TPO mimetic is administered to the subject by subcutaneous injection.

In certain embodiments, the administration of the effective amount of the TPO mimetic results in at least one of a reduced elevation of chemokine KC or alveolar neutrophil infiltration in the subject.

In certain embodiments, the TPO mimetic is RWJ-800088.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows the effect of TPOm-pretreatment (1 day before irradiation at 1 mg/kg body weight by subcutaneous injection) on chemokine (IL-1β, IL-8, TNFα, TGFβ, MCP1, MCP2, KC and MIP2) mRNA expression in the lungs of mice (C57BL/6J male, 9-12 weeks old) at 14 days after whole thorax irradiation (WTI) at 16 Gy delivered by x-ray irradiator. Lung data is presented as fold change of chemokine expression normalized to Hprt1 expression (n=5 for Naïve, n=5 for Vehicle (phosphate buffered saline) and TPOm pretreatment). Expression in the naive group is designated as 1 for comparison. Data are shown as means±SE. *P<0.05 vs. vehicle treated mice.

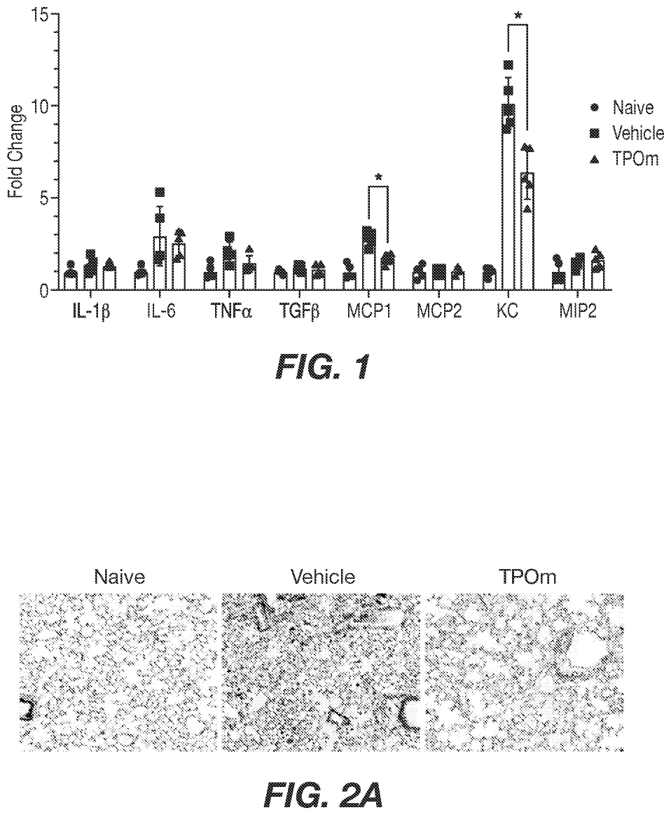

FIGS. 2A-2B show the effect of TPOm-pretreatment (1 day before irradiation at 0.3 mg/kg body weight by subcutaneous injection) on immune cell infiltration and permeability in the lungs of mice at 14 days post-WTI. FIG. 2A shows lung sections of mice were Hematoxylin and Eosin (H&E) stained and visualized for cellularity and immune infiltration. 200× total magnification. FIG. 2B shows the amount of protein in bronchoalveolar lavage measured by bicinchoninic acid assay (BCA) (n=3 for Naïve, n=5 for Vehicle and TPOm pretreatment). *P<0.05 vs. vehicle treated mice.

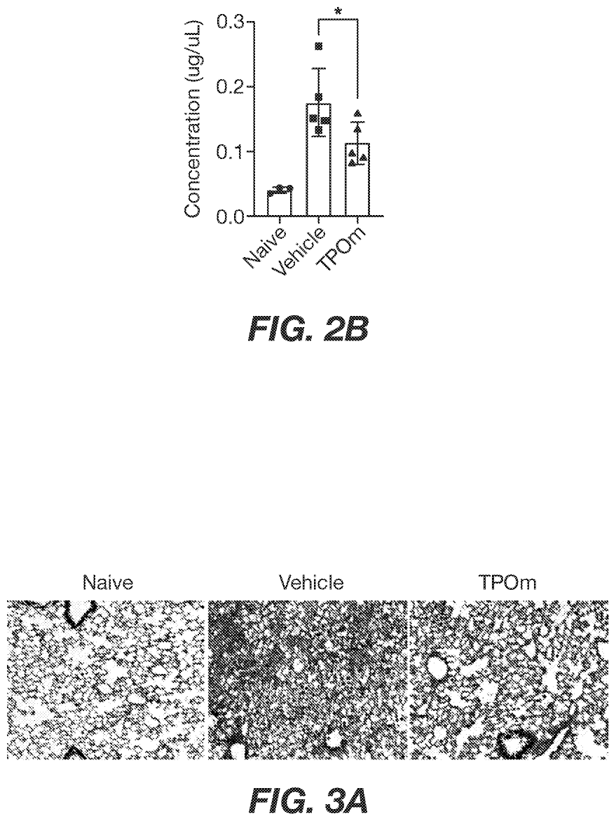

FIGS. 3A-3B show the effect of TPOm-pretreatment (1 day before irradiation at 0.3 mg/kg body weight by subcutaneous injection) on collagen deposition in lungs of mice at 7 months post-WTI. FIG. 3A shows lung sections of mice stained with Trichrome blue and visualized for collagen deposition. 200× total magnification. FIG. 3B shows the percentage of tissue section area positive for collagen (n=3 for Naïve, n=5 for Vehicle and n=5 TPOm pretreatment). *P<0.05 vs. vehicle treated mice.

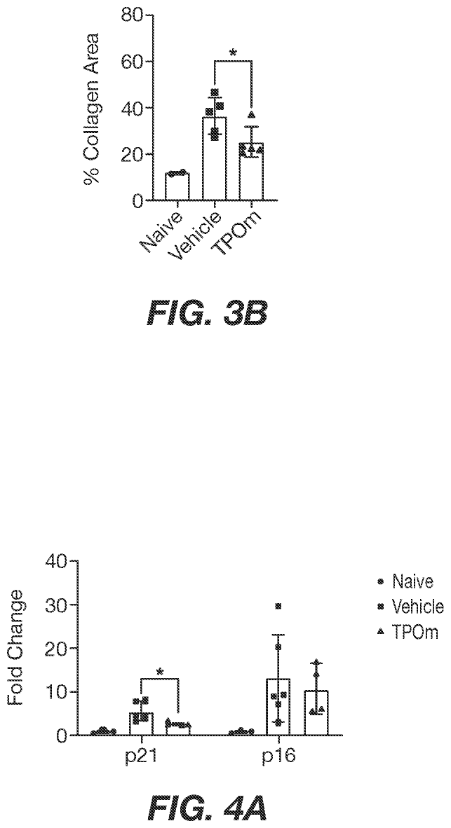

FIGS. 4A-4C show the effect of TPOm-pretreatment (1 day before irradiation at 0.3 mg/kg body weight by subcutaneous injection) on senescence gene mRNA and protein expression in lungs of mice at 7 months post-WTI. mRNA and protein levels assessed by qPCR and western blot, respectively. FIG. 4A shows data presented as fold change of p21 and p16 mRNA expression normalized to Hprt1 expression (n=3 for Naïve, n=5 for Vehicle and n=4 TPOm pretreatment). Data are shown as means±SE. *P <0.05 vs. vehicle treated mice. FIG. 4B shows a representative blot of lung protein lysate. FIG. 4C shows densitometric values of p16 and p21 protein plotted (normalized to β-actin levels) (n=3 for Naïve, Vehicle and TPOm pretreatment). Expression in the naive group is designated as 1 for comparison.

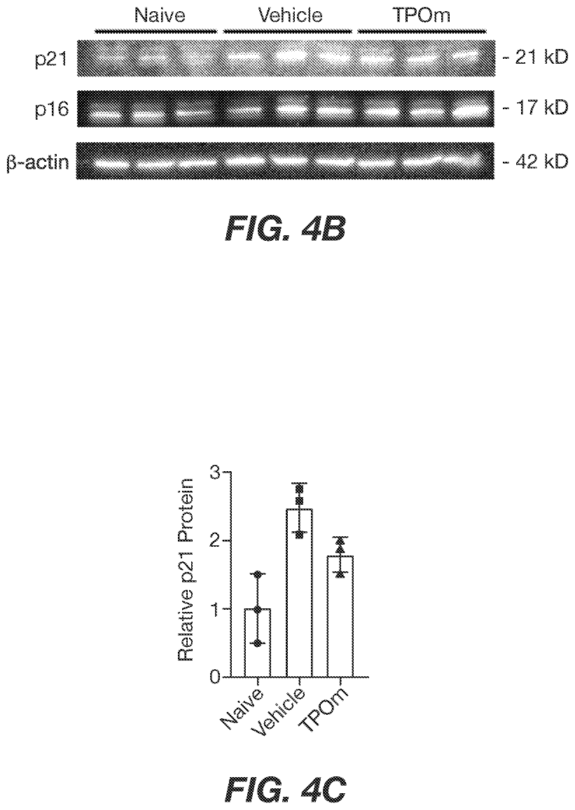

FIGS. 5A-5C show the effect of TPOm-pretreatment (1 day before irradiation at 0.3 mg/kg body weight by subcutaneous injection) on pulmonary density of mice at 7 months post-WTI. Pulmonary density imaged by microCT and measured by microCT analysis software. FIG. 5A shows representative microCT cross section images for each treatment. FIGS. 5B and 5C shows quantification of left lung density longitudinally at months 3, 5 and 7. (n=3 for Naïve, n=5 for vehicle and n=4 for TPOm pretreatment). *P <0.05 vs. vehicle treated mice.

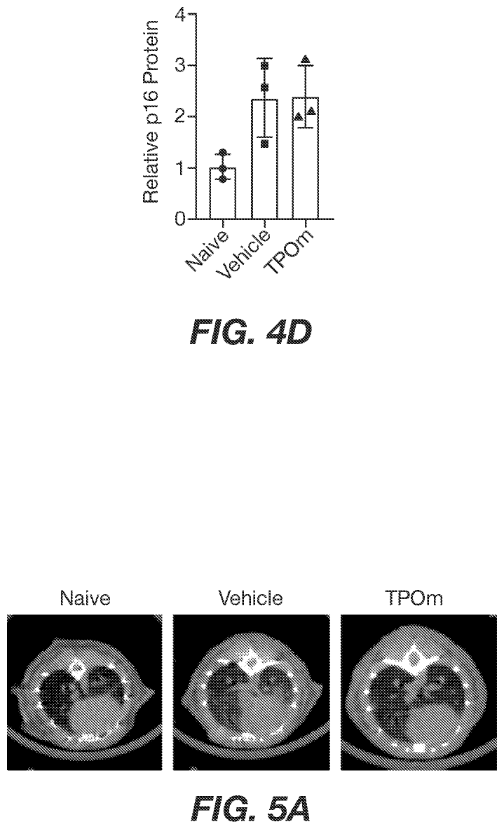

FIG. 6 shows the effect of TPOm-pretreatment on 250-day survival of mice at more lethal dose X-ray radiation (18 Gy) via WTI. Mice were given a vehicle or TPOm injection (1 mg/kg), 1 day before irradiation (1×) or 1 day before irradiation and 7 days after radiation (2×) (n=10 for irradiated groups, n=5 for naïve). *P<0.05 vs. vehicle treated mice.

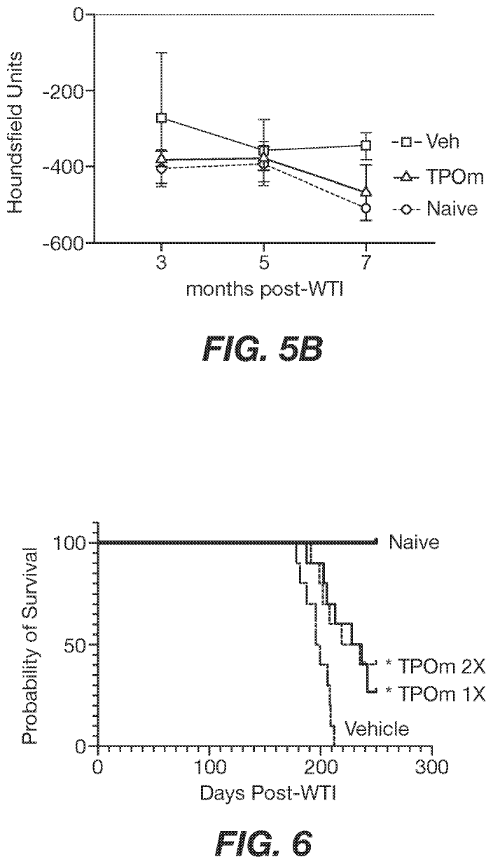

FIGS. 7A-C show TPOm-pretreatment (1 day before irradiation at 0.3 mg/kg body weight by subcutaneous injection) on neutrophil infiltration of mice measured at 7 months post-WTI. FIG. 7A shows quantification of chemokine KC protein in lung lysate measured by ELISA (n=5). FIG. 7B shows representative lung sections of mice stained with myeloperoxidase (MPO). 400× total magnification. FIG. 7C shows quantification of MPO-positive infiltrates in lung as measured by ELISA (n=5). Data are shown as means±SE. *P<0.05, ***P<0.0001.

### DETAILED DESCRIPTION OF THE INVENTION

This disclosure is based, at least in part, on the identification of a thrombopoietin (TPO) mimetic as a therapeutic for mitigating a radiation-induced lung injury in a subject in need thereof. The TPO mimetic can be formulated and administered to the subject who has been, is or will be exposed to radiation to mitigate the radiation-induced lung injury.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human, who will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right”, “left”, “lower” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the term “in combination”, in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

The term “RILT” or “radiation-induced lung injury,” as used herein, refers to an acute response during or within the first few weeks of radiation exposure or radiation therapy (RT) or as a late-response months after radiation exposure or RT. Examples of radiation-induced lung injury (RILI) can include, but are not limited to, radiation-induced pulmonary inflammation, deposition of collagen in the lungs, plural infusion of fluid, and lung fibrosis.

### Radiation Therapy

The term “TRT” or “targeted radiation therapy”, as used herein, refers to a therapy using ionizing radiation, or a radiomimetic agent, that is preferentially targeted or localized to a specific organ or part of the body. It is generally used as part of cancer treatment. TRT, such as targeted ionizing radiation therapy, is sometimes also referred to as radiation treatment, radiotherapy, irradiation, or x-ray therapy. There are three main divisions of targeted ionizing radiation therapy: external beam radiation therapy (EBRT or XRT), internal radiation therapy, and systemic radioisotope therapy. The radiation can be given in several treatments to deliver the same or slightly higher dose, which is called fractioned radiation therapy. As used herein, the term “radiomimetic agent” or “radiomimetic chemical agent” refers to a chemical agent that produces an effect similar to that of ionizing radiation when administered to a subject. Examples of such effect include DNA damage. Examples of radiomimetic chemical agents include, but should not be considered limited to, etoposide, doxorubicin, carboplatin, and bleomycin. Radiomimetic chemical agents such as those described herein can be administered locally to a subject to allow for a targeted application of the agent in a therapeutic manner.

External beam radiation therapy (EBRT) uses a machine that directs high-energy rays from outside the body into the tumor. Current radiation technology allows the precise delivery of external beam radiation therapy, such as targeted radiation therapy which uses computers to create a 3-dimensional picture of the tumor in order to target the tumor as accurately as possible and give it the highest possible dose of radiation while sparing normal tissue as much as possible. Examples of EBRT include, but are not limited to, stereotactic radiation therapy, image guided radiation therapy (IGRT), intensity modulated radiation therapy (IMIRT), helical-tomotherapy, proton beam radiation therapy, and intraoperative radiation therapy (IORT). Among them, stereotactic radiation is a specialized type of external beam radiation therapy. It uses focused radiation beams targeting a well-defined tumor using extremely detailed imaging scans. There are two types of stereotactic radiation: stereotactic radiosurgery (SRS) is for stereotactic radiation treatment of the brain or spine, while stereotactic body radiation therapy (SBRT) refers to more precise targeted radiation treatment to organs within the body such as the lungs.

Internal radiation is also called brachytherapy, in which a radioactive implant is put inside the body in or near the tumor. It allows a higher dose of radiation in a smaller area than might be possible with external radiation treatment. It uses a radiation source that's usually sealed in a small holder called an implant. Different types of implants may be called pellets, seeds, ribbons, wires, needles, capsules, balloons, or tubes. Several such examples of internal radiation are Y-90 SIR-sphere and/or Thera-Sphere.

Targeted systemic radioisotope therapy (SRT) is also called unsealed source radiotherapy. Targeted radioactive drugs are used in SRT to treat certain types of cancer systemically, such as thyroid, bone, and prostate. These drugs, which are typically linked to a targeting entity—such as a monoclonal antibody or a cell-specific ligand, can be given by mouth or put into a vein; they then travel through the body until reaching the desired target, where the drug will accumulate in a relatively high concentration.

As used herein “a subject treated with a targeted radiation therapy” refers to a subject who is undergoing a targeted radiation treatment and the treatment can be before, after or simultaneously with the administration of the TPO mimetic.

### TPO Mimetic

As used herein, a “TPOm”, “TPO mimetic” or “thrombopoietin mimetic” refers to a compound comprising a peptide capable of binding to and activating a thrombopoietin receptor or c-mpl. Preferably, in a TPO mimetic useful for the invention, the peptide capable of binding to and activating a thrombopoietin receptor has no significant homology with thrombopoietin (TPO). The lack of homology with TPO reduces the potential for generation of antibodies against endogenous TPO. Examples of such peptide useful in a TPO mimetic include, but are not limited to, those described in U.S. Publication Nos. 2003/0158116; 2005/0137133; 2006/0040866; 2006/0210542; 2007/0148091; 2008/0119384; U.S. Pat. Nos. 5,869,451; 7,091,311; 7,615,533; 8,227,422; International Patent Publications WO2007/021572; WO2007/094781; and WO2009/148954, the entire contents of which are incorporated herein by reference. More preferably, in a TPO mimetic useful for the invention, the peptide capable of binding to and activating a thrombopoietin receptor is covalently linked to a moiety that improves one or more properties of the peptide. By way of a non-limiting example, the moiety can be a hydrophilic polymer, including but not limited to polyethylene glycol (PEG), polypropylene glycol, polylactic acid and polyglycolic acid. The moiety can also be a polypeptide, such as a Fc region or an albumin.

U.S. Pat. Nos. 7,576,056 and 7,723,295 to Janssen Pharmaceutica NV disclose the use of a TPO compound to treat a patient suffering from thrombocytopenia.

U.S. Pat. Nos. 8,067,367 and 8,283,313 to Janssen Pharmaceutica NV disclose a method of providing hematopoietic stem cells to a subject comprising administering a TPO compound.

U.S. Pat. No. 7,615,533 to Janssen Pharmaceutica NV discloses a method of preventing the development of anemia following treatment selected from the group consisting of treatment with a cytotoxic agent, treatment with an anti-tumor agent and treatment with radiation comprising administering an effective amount of a TPO compound to a subject in need thereof.

U.S. Published Application No. 20200164039 to Janssen Pharmaceutica NV discloses a method of protecting vascular integrity in a subject exposed to a targeted radiation therapy, comprising administering to the subject an effective amount of a TPO compound.

U.S. Published Application No. 20200237870 to Janssen Pharmaceutica NV and Rutgers, The State University discloses a method of mitigating a toxic effect of at least one of a vesicant and a caustic gas in a subject in need thereof, comprising administering to the subject an effective amount of a TPO compound.

U.S. Published Application No. 20200237871 to Janssen Pharmaceutica NV and Montefiore Medical Center discloses a method of mitigating a targeted radiation therapy-induced liver disease in a subject in need thereof, comprising administering to the subject an effective amount of a TPO compound.

U.S. Published Application No. 20200237872 to Janssen Pharmaceutica NV discloses a method of mitigating vascular injury, promoting organ and/or hematopoietic recovery, enhancing survival, and/or protecting against organ and hematopoietic injury in a human subject that is or has been exposed to radiation.

Application Ser. No. 63/261,957 to Janssen Pharmaceutica and Montefiore Medical Center discloses a method of increasing production of at least one of a hematopoietic progenitor cell, a myeloid progenitor cell, an endothelial progenitor cell and an endothelial cell in a non-irradiated subject that comprises administering to the subject an effective amount of a TPO compound.

In a preferred embodiment, a TPO mimetic useful for the invention comprises a peptide having the amino acid sequence of: IEGPTLRQXaaLAARYaa (SEQ ID NO:1), wherein Xaa is tryptophan (W) or β-(2-naphthyl)alanine (referred to herein as “2-Nal”), and Yaa is alanine (A) or sarcosine (referred herein as “Sar”). Preferably, the peptide of SEQ ID NO:1 is covalently linked to a PEG or fused to a Fc domain.

In some embodiments, a TPO mimetic useful for the invention comprises a peptide of SEQ ID NO:1 covalently linked to a PEG, preferably a PEG having an average molecular weight of between about 5,000 to about 30,000 daltons. Preferably, the PEG is selected from the group consisting of monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM). The PEGylation of the peptide leads to a reduced clearance of the compound without loss of potency. See, e.g., U.S. Pat. No. 7,576,056, the entire contents of which are incorporated herein by reference.

In one preferred embodiment, a TPO mimetic useful for the invention is RWJ-800088 or a derivative thereof. As used herein, “RWJ-800088” refers to a 29-mer peptide having two identical 14-mers (SEQ ID NOs: 2 and 5) linked by a lysinamide residue as follows:

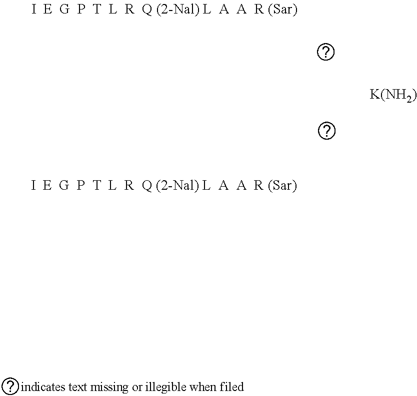

and having a methoxypoly(ethylene glycol) (MPEG) covalently linked to each N-terminal isoleucine, or a pharmaceutically acceptable salt or ester thereof. The RWJ-800088 is thus composed of two 14 amino acid peptide chains of SEQ ID NO:1, where Xaa is 2-Nal and Yaa is Sar, linked by lysinamide reside, and each N-terminal isoleucine is linked to a methoxy polyethylene glycol (MPEG) chain. Accordingly, RWJ-800088 has an abbreviated molecular structure of (MPEG-Ile-Glu-Gly-Pro-Thr-Leu-Arg-Gln-(2-Nal)-Leu-Ala-Ala-Arg-(Sar))2-Lys-NH₂ (SEQ ID NOs: 2 and 5); wherein (2-Nal) is β-(2-naphthyl)alanine, (Sar) is sarcosine and MPEG is methoxypoly(ethylene glycol), or a pharmaceutically acceptable salt or ester thereof. Preferably, the MPEG has an approximately 20,000 Dalton molecular weight or represents methoxypolyethylene glycol20000.

In one embodiment, RWJ-800088 has a molecular structure of formula (I) (SEQ ID NOS 2 and 5, respectively), or a pharmaceutically acceptable salt or ester thereof:

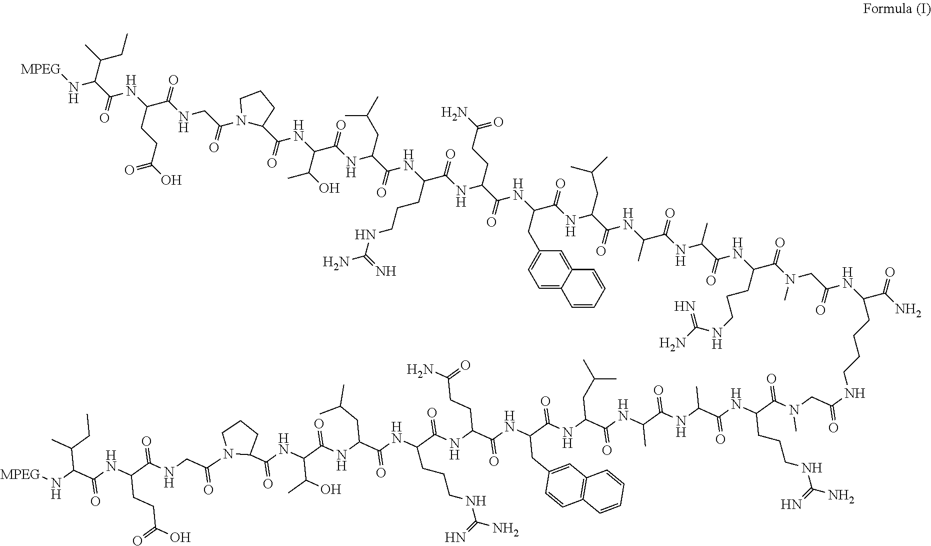

In a preferred embodiment, the MPEG in RWJ-800088 is methoxypolyethyleneglycol20000, and the RWJ-800088 has the full chemical name of: methoxypolyethyleneglycol20000-propionyl-L-Isoleucyl-L-Glutamyl-Glycyl-L-Prolyl-L-Threonyl-L-Leucyl-L-Arginyl-L-Glutaminyl-L-2-Naphthylalanyl-L-Leucyl-L-Alanyl-L-Alanyl-L-Arginyl-Sarcosyl-Ne-(methoxypolyethyleneglycol20000-propionyl-L-Isoleucyl-L-Glutamyl-Glycyl-L-Prolyl-L-Threonyl-L-Leucyl-L-Arginyl-L-Glutaminyl-L-2-Naphthylalanyl-L-Leucyl-L-Alanyl-L-Alanyl-L-Arginyl-Sarcosyl-)-Lysinamide (SEQ ID NOs: 6 and 7), or a pharmaceutically acceptable salt or ester thereof. The molecular weight of the peptide without PEG is 3,295 Daltons and with two 20,000 Dalton MPEG chains is approximately 43,295 Daltons.

In some embodiments, a TPO mimetic useful for the invention comprises a peptide of SEQ ID NO:1 fused to a Fc domain. Fusing the peptide to a Fc domain can stabilize the peptide in vivo. See, e.g., U.S. Pat. No. 6,660,843, the entire contents of which are incorporated herein by reference.

In another preferred embodiment, a TPO mimetic useful for the invention is romiplostim. As used herein, “romiplostim” refers to fusion protein having a Fc domain linked to the N-terminal isoleucine of the peptide of SEQ ID NO:1, where Xaa is W and Yaa is A. In particular, romiplostim has the following amino acid sequence:

(SEQ ID NO: 4)
MDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQV
SLTCLVKGFYPSDIAVEWESNGQPENNYKTTPPVLDSDGSFFLYSKLT
VDKSRWQQGNVFSCSVMHEALHNHYTQKSLSLSPGKGGGGGIEGPTLR
QWLAARAGGGGGGGGIEGPTLRQWLAARA,

It has the thrombopoietin receptor binding domain amino acid sequence of

(SEQ ID NO: 3)
IEGPTLRQWLAARA.

### Dosage and Administration

In the current invention, the inventors discovered that TPO mimetics have significant mitigating effects on RILIs. Thus, methods of the invention comprise administering to a subject in need thereof an effective amount of a TPO mimetic to thereby achieve one or more beneficial results, such as mitigating one or more RILIs, in the subject in need thereof, such as a subject exposed to radiation or treated with a radiation therapy.

The TPO mimetic can, for example, be administered as an active ingredient of a pharmaceutical composition in association with a pharmaceutical carrier or diluent. The TPO mimetics can be administered by oral, pulmonary, parental (intramuscular (IM), intraperitoneal (IP), intravenous (IV) or subcutaneous (SC) injection), inhalation (via a fine powder formulation), transdermal, nasal, vaginal, rectal, or sublingual routes of administration in dosage forms appropriate for each route of administration. Preferably, the TPO mimetic is administered by subcutaneous injection. For example, International Publication No. WO1993/25221 (Bernstein et al.) discloses biodegradable polymer microspheres containing erythropoietin (EPO), which can be administered topically, locally or systemically by parenteral administration or enteral administration, preferably oral administration. WO1994/17784 (Pitt et al.) discloses that EPO can be administered systemically via pulmonary route and that such delivery results in comparable levels of therapeutic benefit as compared with other EPO administration methods. Similar compositions and methods can be used for the administration of TPO mimetic of the present disclosure.

Solid dosage forms for oral administration include capsules, tablets, pills, powders, and granules. In such solid dosage forms, the active peptide compound is admixed with at least one pharmaceutically acceptable carrier such as sucrose, lactose, or starch. Such dosage forms can also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

Preparations for parental administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They may be sterilized by, for example, filtration through bacteria retaining filter, by incorporating sterilizing agents into the compositions, by irradiating the compositions, or by heating the compositions. They can also be manufactured using sterile water, or some other sterile injectable medium immediately before use.

Administration of the TPO mimetic is typically intramuscular, subcutaneous, or intravenous. However other modes of administration such as cutaneous, intradermal or nasal can be envisaged as well. Intramuscular administration of the TPO mimetic can be achieved by using a needle to inject a suspension of the TPO mimetic composition. An alternative is the use of a needleless injection device to administer the composition (using, e.g., Biojector™) or a freeze-dried powder of the TPO mimetic composition.

For intravenous, cutaneous or subcutaneous injection, the TPO mimetic composition can be in the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation can also be employed.

Compositions for rectal or vaginal administration are preferably suppositories which may contain, in addition to the active TPO mimetic, excipients such as cocoa butter or a suppository wax. Compositions for nasal or sublingual administration are also prepared with standard excipients well known in the art.

Typically, administration will have a therapeutic and/or prophylactic aim to mitigate the radiation-induced lung injury in a subject prior to, during or following the radiation therapy. In therapeutic applications, the TPO mimetic compositions are administered to a subject during or after the exposure to radiation therapy, and the TPO mimetic compositions are administered in an amount sufficient to cure or at least partially provide mitigation for the radiation-induced lung injury. In prophylactic applications, TPO mimetic compositions are administered to a subject susceptible to-or at risk of developing RILIs prior to an exposure to radiation and to enhance lung function post-exposure to radiation in a subject in need thereof. In each of these scenarios, the amount of the TPO mimetic compositions will depend on the state and nature of the exposure (e.g., type of radiation therapy, dose and length of exposure), the physical characteristics of the subject (e.g., height, weight, disease state, etc.), and the design of the treatment (e.g., TPOm alone or in combination with another therapeutic agent, etc.)

The pharmaceutically acceptable compositions containing the TPO mimetic are administered to a subject, giving rise to mitigating the radiation-induced lung injury. An amount of a composition sufficient to mitigate the disease is defined to be an “effective dose” or an “effective amount” of the composition.

The actual amount administered, and rate and time-course of administration, will depend on the nature and severity of what is being treated. Prescription of treatment, e.g., decisions on dosage etc., is within the responsibility of general practitioners and other medical doctors, or in a veterinary context a veterinarian, and typically takes account of category and dose of the radiation therapy, the condition of the individual patient, the site of delivery, the method of administration and other factors known to practitioners. Examples of the techniques and protocols mentioned above can be found in Remington's Pharmaceutical Sciences, 16th edition, Osol, A. ed., 1980.

The TPO mimetic can be administered in combination with a targeted radiation therapy.

In certain embodiments, the TPO mimetic is administered to the subject 7 days before to 7 days after, or 2 days before to 2 days after, or simultaneously with a targeted radiation therapy. In certain embodiments, the TPO mimetic is administered to the subject 24 hours before to 24 hours after, or simultaneously with a targeted radiation therapy. For example, the TPO mimetic is administered to the subject 24, 20, 16, 12, 8, 4, 2, 1, 0.5 or 0.1 hours (or any range represented therebetween) before a targeted radiation therapy, or 24, 20, 16, 12, 8, 4, 2, 1, 0.5 or 0.1 hours (or any range represented therebetween) after a targeted radiation therapy. Preferably, the TPO mimetic is administered to the subject about 24 to about 1 hour (or any range represented therebetween) before the subject is administered with a targeted radiation therapy.

Any suitable dose of the radiation can be used in a method of the application in view of the disclosure in the application and the knowledge in the art. To investigate the nature of radiation injury to the host, experiments can also be performed with mice that received TR and TPOm. Animals from various cohorts can be sacrificed at various time points (1 d, 2 d, 3 d, 1 wk, 3 wk, 6 wk and 12 wk) and lung sections can be stained with H&E for histopathological analysis. BrdU and TUNEL staining can be performed for examining lung cell proliferation and apoptosis, respectively.

In certain embodiments, the targeted radiation therapy is targeted radiation to the lung, preferably at a dose of 5-70 Gray (Gy), such as 5, 10, 20, 30, 40, 50, 60, or 70 Gy (or any range represented therebetween), in 1 to 10 fractions, such as 1, 2, 3, 4, 5, 6, 7, 8, 9 or 10 fractions. In other embodiments, the targeted radiation therapy is a preparative IR, preferably a lower dose of IR, more preferably administered in the clinic using stereotactic radiosurgery (SRS) or 3-D conformal TRT (3-D CRT) techniques. In other embodiments, the targeted radiation therapy is partial lung irradiation administered using modern techniques of BIRT to parts of the lung.

Any suitable effective amount of TPOm can be used in a method of the application. Such effective amount can be determined using methods known in the art in view of the present disclosure. In certain embodiments, the effective amount of the TPO mimetic is about 1 to about 10 g/kg, such as 1, 2, 3, 4, 5 or 6 g/kg (or any range represented therebetween), of body weight of the subject. In preferred embodiments, the effective amount of the TPO mimetic is about 1 to about 3 μg/kg of body weight of the subject.

In certain embodiments, the effective amount of the TPO mimetic is administered to the subject by any one of intravenous, intramuscular, intracutaneous, or subcutaneous injection. In a preferred embodiment, the TPO mimetic is administered by subcutaneous injection.

Following production of the TPO mimetic and optional formulation of the TPO mimetic into compositions, the compositions can be administered to an individual, particularly human or another primate. Administration can be to humans, or another mammal, e.g., mouse, rat, hamster, guinea pig, rabbit, sheep, goat, horse, cow, donkey, monkey, dog or cat. Delivery to a non-human mammal need not be for a therapeutic purpose but can be for use in an experimental context.

The TPO mimetic compositions of the invention can be administered alone or in combination with other treatments or additional therapeutic agents, either simultaneously or sequentially dependent upon the condition to be treated.

The term “additional therapeutic agent,” as used herein, refers to any compound or therapeutic agent known to or that demonstrates advantageous properties when administered with a TPO mimetic in a method of the application.

The TPO mimetic compositions can, if desired, be presented in a kit, pack or dispenser, which can contain one or more unit dosage forms containing the active ingredient. The kit, for example, can comprise metal or plastic foil, such as a blister pack. The kit, pack, or dispenser can be accompanied by instructions for administration. The kit can further comprise at least one additional therapeutic agent or a device for mitigating the toxic effect. The kit can further include the additional therapeutic agent, such as those described herein. The device included in the kit can be, for example, a container, a delivery vehicle, or an administration device.

### Lung Function Tests

As used herein, the phrase “lung function” refers to a function of the lung, including, but not limited to, protein synthesis and lung metabolic function.

The lung function can be determined using methods known in the art in view of the present disclosure.

### EMBODIMENTS

The invention provides also the following non-limiting embodiments.

Embodiment 1 is a method of mitigating a radiation-induced lung injury (RILI) in a subject in need thereof, comprising administering to the subject an effective amount of a thrombopoietin (TPO) mimetic, wherein the administration of the effective amount of the TPO mimetic to the subject mitigates the radiation-induced lung injury in the subject.

Embodiment 1(a) is the method of embodiment 1, wherein the TPO mimetic comprises a peptide having the amino acid sequence of SEQ ID NO:1.

Embodiment 1(b) is the method of embodiment 1(a), wherein the peptide has the amino acid sequence of SEQ ID NO:2.

Embodiment 1(c) is the method of embodiment 1(a) or 1(b), wherein the TPO mimetic further comprises a hydrophilic polymer covalently linked to the peptide.

Embodiment 1(d) is the method of embodiment 1(c), wherein the hydrophilic polymer is any one of: i) polyethylene glycol (PEG), ii) polypropylene glycol, iii) polylactic acid, or iv) polyglycolic acid.

Embodiment 1(e) is the method of embodiment 1(d), wherein the hydrophilic polymer is PEG.

Embodiment 1(f) is the method of embodiment 1(e), wherein the PEG is any one of monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), or monomethoxypolyethylene glycol-imidazolyl-carbonyl (MePEG-IM).

Embodiment 1(g) is the method of embodiment 1(e), wherein the PEG is methoxypoly(ethylene glycol) (MPEG).

Embodiment 1(h) is the method of embodiment 1(g), wherein the TPO mimetic is RWJ-800088 having a molecular structure of formula (I), or a pharmaceutically acceptable salt or ester thereof.

Embodiment 1(i) is the method of embodiment 1(h), wherein the MPEG in the RWJ-800088 is methoxypolyethylene glycol20000.

Embodiment 1(j) is the method of embodiment 1(a), wherein the peptide has the amino acid sequence of SEQ ID NO:3.

Embodiment 1(k) is the method of embodiment 1(j), wherein the peptide is fused to a polypeptide.

Embodiment 1(1) is the method of embodiment 1(k), wherein the polypeptide is a Fc domain.

Embodiment 1(m) is the method of embodiment 1(1), wherein the TPO mimetic is romiplostim.

Embodiment 1(m)(1) is the method of embodiment 1(m), wherein romiplostim comprises the amino acid sequence of SEQ ID NO:4.

Embodiment 2 is the method of any one of embodiments 1 to 1(m)(1), wherein the radiation-induced lung injury is any one of more of the following diseases: radiation-induced pulmonary inflammation, deposition of collagen in the lungs, plural infusion of fluid, and lung fibrosis.

Embodiment 2(a) is the method of embodiment 2, wherein the radiation-induced lung injury is elevated lung enzymes.

Embodiment 3 is the method of any one of embodiments 1 to 1(m)(1), wherein the subject is treated with a targeted radiation therapy for a lung disease.

Embodiment 3(a) is the method of embodiment 3, wherein the subject is treated with a stereotactic radiation therapy.

Embodiment 3(b) is the method of embodiment 3, wherein the subject is treated with a transarterial chemoembolization (TACE).

Embodiment 3(c) is the method of embodiment 3, wherein the lung disease is a lung tumor.

Embodiment 3(d) is the method of embodiment 3, wherein the lung disease is a lung metastasis.

Embodiment 3(e) is the method of embodiment 3, wherein the lung disease is a lung cancer.

Embodiment 3(f) is the method of embodiment 3, wherein the lung disease is a genetic disorder.

Embodiment 3(g) is the method of embodiment 3(e), wherein the genetic disorder results in a protein deficiency.

Embodiment 4 is the method of any one of embodiments 1 to 1(m)(1), wherein the subject is treated with a preparative irradiation for bone marrow transplant.

Embodiment 4 is the method of any one of embodiments 1 to 1(m)(1), wherein the TPO mimetic is administered to the subject at least about 2 days before to at least about 2 days after the subject is administered a dose of targeted radiation.

Embodiment 4(a) is the method of embodiment 4, wherein the TPO mimetic is administered to the subject at least about 24 hours before to at least about 24 hours after the subject is administered a dose of targeted radiation.

Embodiment 4(b) is the method of embodiment 4, wherein the TPO mimetic is administered to the subject 24 hours to 2 hours after the subject is administered the dose of radiation.

Embodiment 4(c) is the method of the method of embodiment 4, wherein the TPO mimetic is administered to the subject 24 hours before the subject is administered the dose of radiation.

Embodiment 4(d) is the method of embodiment 4, wherein the TPO mimetic is administered to the subject 23 hours before the subject is administered the dose of radiation.

Embodiment 4(d) is the method the method of embodiment 4, wherein the TPO mimetic is administered to the subject 22 hours before the subject is administered the dose of radiation.

Embodiment 4(e) is the method of embodiment 4, wherein the TPO mimetic is administered to the subject 21 hours before the subject is administered the dose of radiation.

Embodiment 4(f) is the method of embodiment 4, wherein the TPO mimetic is administered to the subject 20 hours before the subject is administered the dose of radiation.

Embodiment 4(g) is the method of embodiment 4, wherein the TPO mimetic is administered to the subject 19 hours before the subject is administered the dose of radiation.

Embodiment 4(h) is the method of embodiment 4, wherein the TPO mimetic is administered to the subject 18 hours before the subject is administered the dose of radiation.

Embodiment 4(i) is the method of embodiment 4, wherein the TPO mimetic is administered to the subject 17 hours before the subject is administered the dose of radiation.

Embodiment 4(j) is the method of embodiment 4, wherein the TPO mimetic is administered to the subject 16 hours before the subject is administered the dose of radiation.

Embodiment 4(k) is the method of embodiment 4, wherein the TPO mimetic is administered to the subject 15 hours before the subject is administered the dose of radiation.

Embodiment 4(1) is the method of embodiment 4, wherein the TPO mimetic is administered to the subject 14 hours before the subject is administered the dose of radiation.

Embodiment 4(m) is the method of embodiment 4, wherein the TPO mimetic is administered to the subject 13 hours before the subject is administered the dose of radiation.

Embodiment 4(n) is the method of embodiment 4, wherein the TPO mimetic is administered to the subject 12 hours before the subject is administered the dose of radiation.

Embodiment 4(o) is the method of embodiment 4, wherein the TPO mimetic is administered to the subject 11 hours before the subject is administered the dose of radiation.

Embodiment 4(p) is the method of embodiment 4, wherein the TPO mimetic is administered to the subject 10 hours before the subject is administered the dose of radiation.

Embodiment 4(q) is the method of embodiment 4, wherein the TPO mimetic is administered to the subject 9 hours before the subject is administered the dose of radiation.

Embodiment 4(r) is the method of embodiment 4, wherein the TPO mimetic is administered to the subject 8 hours before the subject is administered the dose of radiation.

Embodiment 4(s) is the method of embodiment 4, wherein the TPO mimetic is administered to the subject 7 hours before the subject is administered the dose of radiation.

Embodiment 4(t) is the method of embodiment 4, wherein the TPO mimetic is administered to the subject 6 hours before the subject is administered the dose of radiation.

Embodiment 4(u) is the method of embodiment 4, wherein the TPO mimetic is administered to the subject 5 hours before the subject is administered the dose of radiation.

Embodiment 4(v) is the method of embodiment 4, wherein the TPO mimetic is administered to the subject 4 hours before the subject is administered the dose of radiation.

Embodiment 4(w) is the method of embodiment 4, wherein the TPO mimetic is administered to the subject 3 hours before the subject is administered the dose of radiation.

Embodiment 4(x) is the method of embodiment 4, wherein the TPO mimetic is administered to the subject 2 hours before the subject is administered the dose of radiation.

Embodiment 4(y) is the method of embodiment 4, wherein the TPO mimetic is administered to the subject 1 hours before the subject is administered the dose of radiation.

Embodiment 4(z) is the method of embodiment 4, wherein the TPO mimetic is administered to the subject 30 minutes before the subject is administered the dose of radiation.

Embodiment 4(a)(i) is the method of embodiment 4, wherein the TPO mimetic is administered to the subject 15 minutes before the subject is administered the dose of radiation.

Embodiment 4(a)(ii) is the method of embodiment 4, wherein the TPO mimetic is administered to the subject 0.1 to 2 hours after the subject is administered the dose of radiation.

Embodiment 5 is the method of any one of embodiments 1 to 1(m)(1), wherein the dose of the radiation is 5-70 Gray (Gy).

Embodiment 5(a) is the method of embodiment 5, wherein the dose of the radiation is 10 Gray (Gy).

Embodiment 5(b) is the method of embodiment 5, wherein the dose of the radiation is 20 Gray (Gy).

Embodiment 5(c) is the method of embodiment 5, wherein the dose of the radiation is 30 Gray (Gy).

Embodiment 5(d) is the method of embodiment 5, wherein the dose of the radiation is 40 Gray (Gy).

Embodiment 5(e) is the method of embodiment 5, wherein the dose of the radiation is 50 Gray (Gy).

Embodiment 5(f) is the method of embodiment 5, wherein the dose of the radiation is 60 Gray (Gy).

Embodiment 5(g) is the method of embodiment 5, wherein the dose of the radiation is 70 Gray (Gy).

Embodiment 5(h) is the method of any one of embodiments 5-5(g), wherein the dose of the radiation is administered to the subject in 1 to 10 fractions.

Embodiment 6 is the method of any one of embodiments 1 to 1(m)(1), wherein the effective amount of the TPO mimetic is about 1 to about 10 g/kg of body weight of the subject.

Embodiment 6(a) is the method of embodiment 6, wherein the effective amount of the TPO mimetic is about 1 to about 5 μg/kg of body weight of the subject.

Embodiment 6(b) is the method of embodiment 6, wherein the effective amount of the TPO mimetic is about 1 μg/kg of body weight of the subject.

Embodiment 6(c) is the method of embodiment 6, wherein the effective amount of the TPO mimetic is about 3 μg/kg of body weight of the subject.

Embodiment 6(d) is the method of embodiment 6, wherein the effective amount of the TPO mimetic is about 5 μg/kg of body weight of the subject.

Embodiment 7 is the method of any one of embodiments 1 to 1(m)(1), wherein the effective amount of the TPO mimetic is administered to the subject by any one of intravenous, intramuscular, intracutaneous, or subcutaneous injection.

Embodiment 7(a) is the method of embodiment 7, wherein the effective amount of the TPO mimetic is administered to the subject by subcutaneous injection.

Embodiment 7(b) is the method of embodiment 7, wherein the effective amount of the TPO mimetic is administered to the subject by intravenous injection.

Embodiment 7(c) is the method of embodiment 7, wherein the effective amount of the TPO mimetic is administered to the subject by intramuscular injection.

Embodiment 7(d) is the method of embodiment 7, wherein the effective amount of the TPO mimetic is administered to the subject by intracutaneous injection.

Embodiment 8 is a kit of mitigating a radiation-induced lung disease in a subject in need thereof in accordance with is the method of any one of embodiments 1 to 1(m)(1), comprising a pharmaceutical composition comprising an effective amount of a TPO mimetic and a pharmaceutically acceptable carrier.

Embodiment 8(a) is the kit of embodiment 8, further comprising at least one additional therapeutic agent or device for mitigating the radiation-induced lung injury.

Embodiment 9 is a method for treating radiation pneumonitis in a subject in need thereof, comprising administering to the subject a thrombopoietin (TPO) mimetic comprising the amino acid sequence of SEQ ID NO:1, preferably the TPO mimetic is RWJ-800088 or romiplostim, in an amount effective to treat the radiation pneumonitis.

Embodiment 9(a) is the method of embodiment 20, wherein the subject is treated with a targeted radiation therapy for a lung disease or the subject is treated with a preparative irradiation for bone marrow transplant.

Embodiment 9(b) is the method of embodiment 9(b), wherein the targeted radiation therapy is selected from the group consisting of stereotactic radiation therapy and transarterial chemoembolization (TACE).

Embodiment 9(d) is the method of embodiment 9(b) or (c), wherein the subject is treated with the targeted radiation therapy at a dose of 5-70 Gray (Gy) in 1 to 10 fractions.

Embodiment 9(e) is the method of any one of embodiments 9-9(d), wherein the subject is treated for a lung tumor or a lung metastasis, preferably a lung cancer.

Embodiment 9(f) is the method of any one of embodiments 9-9(e), wherein the TPO mimetic is administered to the subject 7 days before to 7 days after; 2 days before to 2 days after; 24 hours before to 24 hours after, preferably about 2 hours to 24 hours before or after, the subject is administered a dose of radiation.

Embodiment 9(g) is the method of embodiment 9(f), wherein the TPO mimetic is administered to the subject 2 hours to 36 hours or 1 day before the subject is administered a dose of radiation.

Embodiment 9(h) is the method of any one of embodiments 9-9(g), wherein the TPO mimetic is administered to the subject by any one of intravenous, intramuscular, intracutaneous, or subcutaneous injection.

Embodiment 9(i) is the method of embodiment 9(h), wherein the TPO mimetic is administered to the subject by subcutaneous injection.

Embodiment 9(j) is the method of any one of embodiments 9-9(h), wherein the administration of the effective amount of the TPO mimetic results in at least one of a reduced elevation of chemokine KC or alveolar neutrophil infiltration in the subject.

Embodiment 9(k) is the method of any one of embodiments 1-9, wherein the TPO mimetic is RWJ-800088.

### EXAMPLES

### Example 1

Thrombopoietin-Mimetic Provides Radioprotection and Prevents Lung Fibrosis in Mice after Whole Thoracic Radiation

Although radiation therapy is an important treatment modality for thoracic cancers, ionizing radiation damage to lung tissue remains to be a dose limiting factor. Radiation-induced lung injury (RILI) involves an acute, inflammatory stage with high immune cell recruitment and infiltration; followed by a late, fibrosis stage with excessive collagen deposition and cellular senescence. Currently there are no drugs approved to treat RILI. TPOm has been shown to provide radioprotection in bone marrow and survival benefits in mice after total body irradiation. The present inventors evaluated the effectiveness of TPOm at reducing the acute and delayed effects of RILI.

Methods: C57BL/6 male mice (9-12 weeks old) were administered with the TPOm RWJ-800088 (0.3 mg/kg) or PBS (vehicle) subcutaneously 1 day prior to whole thoracic radiation (WTI) at 16 Gy delivered by x-ray. Mice were sacrificed at 2 weeks and 7 months (n=5/time point/group) post-WTI to collect the lung tissues for various analysis. Groups of mice (n=10/group) exposed to 18 Gy WTI were also subjected to an 8-month survival study.

Results: At 2 weeks post-WTI, the mRNA expression of proinflammatory chemokines MCP1 and KC in lungs of TPOm treated mice were 39% and 37% lower than vehicle, respectively (p<0.05). The H&E staining showed higher cellularity in vehicle compared to TPOm treated mice. Further, at this timepoint, TPOm treated mice had 28% less protein leaked to bronchoalveolar lavage fluid compared to the vehicle (p<0.05). At the 7-month, mice treated with TPOm had 31% lower collagen deposition than the vehicle judged by Trichrome Blue staining (p<0.05). This result is complimented by microCT analysis showing 21% less density in lungs of mice treated with TPOm, compared to vehicle (p<0.05). The mRNA levels of senescence marker p21 were also significantly 52% lower in TPOm treated mice than the vehicle (p<0.05). In the 8-month survival study, mice treated with TPOm had a significantly delayed in mortality and increased survival from 0% in the vehicle to 40% (p<0.01).

Conclusion: TPOm decreases vascular leakage, inflammation, fibrosis, and senescence, resulting in an improvement of the survival of WTI mice. Thus, TPOm appears to be a potential regimen to treat RILI.

These results suggest that TPOm and romiplostim have a regenerative effect on lung following IR.

### Example 2

Thrombopoietin-Mimetic Prevents Neutrophil Infiltration in Mice after Whole Thoracic Radiation

Neutrophils are innate immune cells that infiltrate into tissue acutely after damage. Higher neutrophil infiltration is associated with more severe inflammation. To properly assess the anti-inflammatory effect of TPOm on radiation pneumonitis, neutrophil recruitment chemokine, KC, and alveolar neutrophil infiltration were measured.

Methods: Mice were treated as described in Example 1. Whole lung lysate was prepared from left lung tissue at the time of harvest. KC ELISA kit (R&D Systems, #DY453) was used according to manufacturer's instructions. For each sample, 2 replicates were measured for KC protein.

At the time of harvest, the right lung was fixed with 10% formalin, then embedded and sectioned at Sum. Anti-myeloperoxidase (MPO) antibody was obtained from Abcam (Cambridge, MA, USA). Immunoreactivity was visualized by ImmPACT histochemistry (Vector Labs, Burlingame, CA, USA) with hematoxylin counter staining. For quantitation of MPO+ cells in lung tissue, images were analyzed with ImageJ software (National Institutes of Health); stained cells were counted using the “analyze particles” module to assess positive staining. Data were quantified from 3 fields of 1 section per animal, and 5 animals per group.

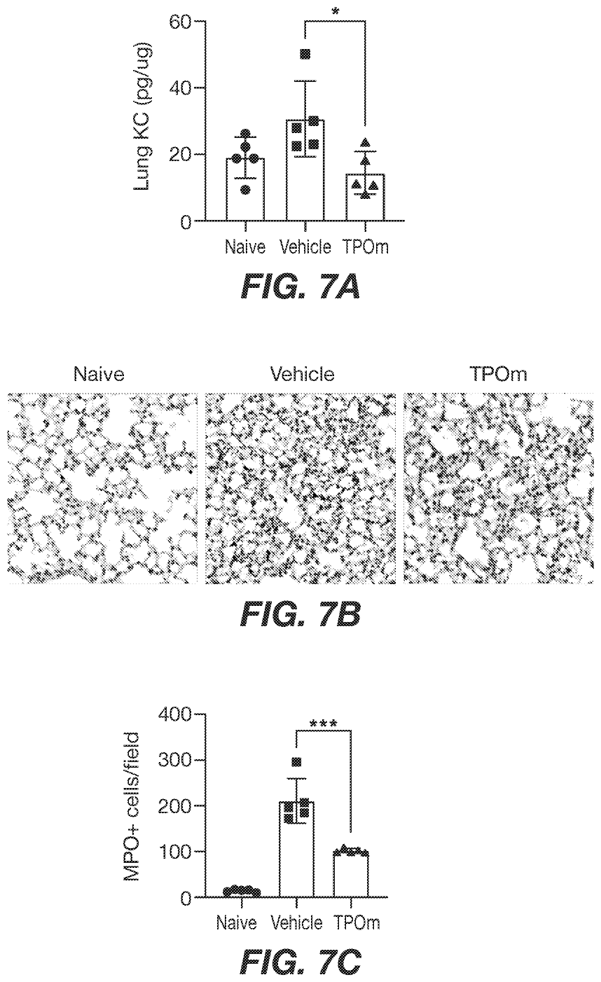

Results: In parallel to mRNA expression, the protein expression of KC in the lung was also significantly reduced in the TPOm (RWJ-800088) group to a level similar to naïve (FIG. 7A). As KC is a major recruitment chemokine for neutrophils, MPO staining was used to assess neutrophil infiltration. Lung sections of the vehicle group showed alveolar neutrophil accumulation in significantly greater numbers than mice treated with TPOm (FIG. 7C).

It will be appreciated by those skilled in the art that changes could be made to the embodiments described without departing from the broad inventive concept thereof. It is understood, therefore, that this invention is not limited to the particular embodiments disclosed, but it is intended to cover modifications within the spirit and scope of the present invention as defined by the present description.

1. A method of mitigating radiation-induced or radiomimetic agent-induced lung injury in a subject in need thereof, the method comprising: administering to the subject an effective amount of a thrombopoietin (TPO) mimetic comprising the amino acid sequence of SEQ ID NO:1.

2. The method of claim 1, wherein the radiation-induced or radiomimetic agent-induced lung injury is any one or more of pneumonopathy (pneumonitis) and lung fibrosis, radiation-induced pulmonary inflammation, deposition of collagen in the lungs, plural infusion of fluid, and lung fibrosis.

3. The method of claim 1, wherein the TPO mimetic is selected from RWJ-800088 and romiplostim.

4. The method of claim 1, wherein the subject is treated with a targeted radiation therapy for a lung disease.

5. The method of claim 4, wherein the targeted radiation therapy is selected from the group consisting of stereotactic radiation therapy and transarterial chemoembolization (TACE).

6. The method of claim 4, wherein the subject is treated with targeted radiation at a dose of 5-70 Gray (Gy) in 1 to 10 fractions.

7. The method of claim 1, wherein the subject is treated for a lung tumor or a lung metastasis, preferably a lung cancer.

8. The method of claim 1, wherein the subject is treated with a radiation therapy for a cancer or the subject is treated with a preparative irradiation for bone marrow transplant.

9. The method of any claim 1, wherein the TPO mimetic is administered to the subject 7 days before to 7 days after; 2 days before to 2 days after; 24 hours before to 24 hours after, preferably about 2 hours to 24 hours before or after, the subject is administered a dose of radiation.

10. The method of claim 1, wherein a therapeutically effective amount of the TPO mimetic is administered to the subject.

11. The method of claim 1, wherein the TPO mimetic is administered to the subject by any one of intravenous, intramuscular, intracutaneous, or subcutaneous injection.

12. The method of any claim 1, wherein the administration of the effective amount of the TPO mimetic results in at least one of an increased lung capacity of non-irradiated lobe of lung and a reduced elevation of a circulating lung injury marker in the subject.

13. (canceled)

14. A method for treating a subject with a radiation-induced lung injury comprising administering to the subject after exposure a thrombopoietin (TPO) mimetic comprising the amino acid sequence of SEQ ID NO:1, preferably the TPO mimetic is RWJ-800088 or romiplostim, in an amount effective to treat a radiation-induced lung injury occurring at least 3 months after exposure to radiation.

15. The method of claim 14, wherein the subject is a patient who is receiving radiation therapy, a nuclear power plant worker, a nuclear warfare personnel, or a subject who is exposed to elevated levels of radiation due to a nuclear accident, war or terrorist attack.

16. The method of claim 14, wherein the radiation-induced lung injury occurs at least 6 months after exposure to radiation.

17. The method of claim 14, wherein the thrombopoietin (TPO) mimetic inhibits one or more of radiation-induced pulmonary inflammation, deposition of collagen in the lungs, plural infusion of fluid, and lung fibrosis.

18. The method of claim 14, wherein the subject is a human.

19. The method according to claim 14, wherein the radiation-induced toxicity is radiation-induced lung injury consisting of radiation pneumonitis in the early stage of radiotherapy and radiation pulmonary fibrosis in the late stage of radiotherapy.

20. A method for treating radiation pneumonitis in a subject in need thereof, comprising administering to the subject a thrombopoietin (TPO) mimetic comprising the amino acid sequence of SEQ ID NO:1, preferably the TPO mimetic is RWJ-800088 or romiplostim, in an amount effective to treat the radiation pneumonitis.

21. The method of claim 20, wherein the subject is treated with a targeted radiation therapy for a lung disease or the subject is treated with a preparative irradiation for bone marrow transplant.

22.-30. (canceled)


SEQUENCE LISTING (7 sequences)

SEQ ID NO: 1 - protein, 14 aa, synthetic construct
  VARIANT 9: note = W or beta-(2-naphthyl)alanine
  VARIANT 14: note = A or sarcosine
IEGPTLRQXLAARX

SEQ ID NO: 2 - protein, 15 aa, synthetic construct
  SITE 1: note = Methoxypoly(ethylene glycol) modified residue
  SITE 9: note = beta-(2-naphthyl)alanine
  SITE 14: note = Sarcosine
  SITE 15: note = Amidated residue
IEGPTLRQXLAARXK

SEQ ID NO: 3 - protein, 14 aa, synthetic construct
IEGPTLRQWLAARA

SEQ ID NO: 4 - protein, 269 aa, synthetic construct [sequence omitted]

SEQ ID NO: 5 - protein, 14 aa, synthetic construct
  SITE 1: note = Methoxypoly(ethylene glycol) modified residue
  SITE 9: note = beta-(2-naphthyl)alanine
  SITE 14: note = Sarcosine
IEGPTLRQXLAARX

SEQ ID NO: 6 - protein, 14 aa, synthetic construct
  SITE 1: note = Methoxypolyethyleneglycol20000-propionyl modified residue
  SITE 9: note = Beta-(2-naphthyl)alanine
  SITE 14: note = N-Lauroylsarcosine
IEGPTLRQXLAARX

SEQ ID NO: 7 - protein, 15 aa, synthetic construct
  SITE 1: note = Methoxypolyethyleneglycol20000-propionyl modified residue
  SITE 9: note = Beta-(2-naphthyl)alanine
  SITE 14: note = N-Lauroylsarcosine
  SITE 15: note = Amidated residue
IEGPTLRQXLAARXK